(19) United States
(12) Patent Application Publication
(10) Pub. No.: US 2020/0254089 A1
(43) Pub. Date: Aug. 13, 2020

(54) VACCINE COMPOSITION AND ADJUVANT

(71) Applicant: Atomis Inc., Kyoto (JP)

(72) Inventors: Daisuke ASARI, Kyoto (JP); Shinji KATO, Kyoto (JP)

(21) Appl. No.: 16/616,957

(22) PCT Filed: Jun. 6, 2018
(86) PCT No.: PCT/JP2018/021695

§ 371 Date: Nov. 25, 2019

(30) Foreign Application Priority Data
Jun. 6, 2017 (JP) 2017112115

(51) Int. Cl. A61K 39/39; C07K 16/00

(52) U.S. Cl. CPC A61K 39/39; A61K 9/0019; C07K 16/00

(57) ABSTRACT

An object of the present invention is to provide an excellent vaccine composition and adjuvant. The vaccine composition according to the present invention includes an antigen for inducing immunity and a Metal Organic Framework (MOF). The adjuvant according to the present invention includes a MOF.

### CROSS-REFERENCE TO RELATED APPLICATIONS

This is a 371 application of International Patent Application Number PCT/JP2018/021695 filed Jun. 6, 2018 claiming priority from Japanese Patent Application Number JP2017-112115 filed Jun. 6, 2017, and the disclosures of which are incorporated herein by reference in their entirety.

### TECHNICAL FIELD

The present invention relates to vaccine compositions and adjuvants.

### BACKGROUND ART

Various vaccine compositions have conventionally been used for the prevention or treatment of infectious diseases. Adding adjuvants to vaccine compositions is a common practice for reinforcing their antigenicity.

On the other hand, a group of materials called Metal Organic Framework (MOF) or Porous Coordination Polymer (PCP) has attracted attention in such fields as gas separation, which are distant from the community of immunology. The MOFs typically form a porous structure by combination of a metal and a multidentate ligand.

### CITATION LIST

### Patent Literature

[Patent Literature 1] WO2004/037895
[Patent Literature 2] WO2009/042802

### Non-Patent Literature

[Non-Patent Literature 1] David Farrusseng, Metal-Organic Frameworks: Applications from Catalysis to Gas Storage, Wiley, 2011
[Non-Patent Literature 2] Yabing He et al. Methane Storage in Metal-Organic Frameworks, Chem Soc Rev., 2014

### SUMMARY OF THE INVENTION

### Technical Problem

An object of the present invention is to provide an excellent vaccine composition and adjuvant.

### Solution to Problem

Some aspects of the present invention are as described below.

[1] A vaccine composition comprising an antigen for inducing immunity and a Metal Organic Framework (MOF).
[2] The vaccine composition according [1], further comprising an immune signal transducer.
[3] The vaccine composition according to [2], wherein at least a part of the immune signal transducer is contained in pores of the MOF.
[4] The vaccine composition according to [3], wherein the MOF is configured to decompose in vivo to release at least a part of the immune signal transducer.
[5] The vaccine composition according to any one of [2]-[4], wherein the immune signal transducer is a small molecule having a molecular weight of 1000 or less.
[6] The vaccine composition according to [5], wherein the immune signal transducer is a gas at 25° C. and 100 kPa.
[7] The vaccine composition according to any one of [2]-[6], wherein the immune signal transducer is a factor that is configured to act on keratinocytes, monocytes, lymphocytes, or granulocytes.
[8] The vaccine composition according to any one of [1]-[7], wherein the MOF comprises at least one metal element selected from the group consisting of calcium, magnesium, iron, zinc, aluminum, potassium, and sodium.
[9] The vaccine composition according to any one of [1]-[8], wherein the vaccine composition is configured to be administered on a skin and/or a mucous membrane.
[10] The vaccine composition according to any one of [1]-[8], wherein the vaccine composition is configured to be administered by an intradermal injection, a subcutaneous injection, or an intramuscular injection.
[11] An adjuvant comprising a Metal Organic Framework (MOF).
[12] The adjuvant according to [11], wherein the MOF contains an immune signal transducer in its pores.
[13] The adjuvant according to [12], wherein the MOF is configured to decompose in vivo to release at least a part of the immune signal transducer.

### Advantageous Effects of Invention

The present invention makes it possible to provide an excellent vaccine composition and adjuvant.

### BRIEF DESCRIPTION OF DRAWINGS

FIG. 1A is a CO adsorption profile of a metal organic framework AP004 [MIL-100 (Fe)].

FIG. 1B is a NO adsorption profile of a metal organic framework AP004 [MIL-100 (Fe)].

FIG. 2 is a NO adsorption profile of a metal organic framework AP104 (BioMIL-3).

FIG. 3 is a graph showing the results of measurement of antigen-specific antibody titers in mouse serum.

FIG. 4A is a graph showing the results of measurement of OVA-specific cytokine production.

FIG. 4B is a graph showing the results of measurement of OVA-specific cytokine production.

### DESCRIPTION OF EMBODIMENTS

Vaccine compositions and adjuvants according to an embodiment of the present invention are hereinafter described.

The vaccine composition according to the present disclosure includes an antigen for inducing immunity and a Metal Organic Framework (MOF). The MOF mainly functions as an adjuvant in the composition.

The antigens can be any substances that may induce an immune response. For instance, the antigens can be proteins or peptides. Using an antigen with a low molecular weight is commonly preferred in transdermal administration where the skin permeability of the antigen is required. A peptide with about 8 to 12 amino acids may thus be preferably used in such an occasion. Other antigens such as cancer antigen peptide or an antigen derived from an infectious pathogen can also be used.

Alternatively, autoantigens (for example, antigens related to autoimmune diseases), endogenous antigens (for example, antigens derived from cancer), foreign antigens (for example, antigens related to allergies or antigens derived from viruses and bacteria), or other antigens can also be used.

Examples of the antigens related to autoimmune diseases include:

Amyloid β, which is believed to cause Alzheimer's disease, or its precursors, or its fragment proteins or peptides;

α-synuclein, which is believed to cause Parkinson's disease, or its fragment proteins or peptides;

α-fodrin, which is believed to cause Sjogren's syndrome, or its fragment proteins or peptides;

Thyroid hormone receptor, which is believed to cause Graves' disease, or its fragment proteins or peptides;

Ganglioside, which is believed to cause Guillain-Barre syndrome, or its fragment proteins or peptides;

DNA or its fragments, which is believed to cause systemic lupus erythematosus; Cholesterol ester transfer protein, apolipoprotein, or oxidized LDL, which are believed to cause arteriosclerosis, or their fragment proteins or peptides;

Angiotensin I/II, which is believed to cause high blood pressure, or its fragment proteins or peptides;

Insulin, GAD, or IL-1β, which are believed to cause type 1 diabetes, or their fragment proteins or peptides;

Acetylcholine receptor, which is believed to cause myasthenia gravis, or its fragment proteins or peptides;

TNFα or IL-6, which are believed to cause chronic rheumatoid arthritis, or their fragment proteins or peptides; and

TRANCE or RANKL, which are believed to cause osteoporosis, or their fragment proteins or peptides.

Examples of the antigens derived from cancer include WT1, PR1, GPC3, HER-2, MAGE-A1, MAGE-A2, MAGE-A3, tyrosinase, gp100, CEA, hTRT, EGF receptor, mTERT, PRAME, PSMA, PSA-1, cytochrome p450, NY-ESO-1, Survivine, MUC-1, MAGE-A10, or PAP, or proteins or peptides derived therefrom.

Examples of the Antigens Related to Allergies Include:

Allergens derived from trees (e.g. acacia, alder tree, velvet blue radish, beech, birch, maple, mountain cedar, red cedar, boxwood, cypress, American elm, Chinese elm, pseudotsuga japonica, rubber, eucalyptus, Japanese hackberry, hickory, American linden, sugar maple, mesquite, paper mulberry, konara oak, olive, pecan, pepper, pine, privet, Russian olive, American sycamore, elderberry, black walnut, or black willow);

Allergens derived from vegetations (e.g. cotton, gypsy moth, Kentucky bluegrass, bromus japonicus, corn, meadow fescue, Johnson grass, oats, moths, knuckles, barley, rice, vernal grass, timothy, amaranthaceae, red-tailed geese, red-tailed eels, red-tailed geese, tall goldenrod, kochia [firebush], lambs quarters, calendula, nettle, rough pigweed, English plantain, tall ragweed, short ragweed, false ragweed, Russian thistle, common sagebrush, licorice, or sheep sorrel);

Allergens derived from insects (e.g. silkworms, ticks, bees, wasps, ants, or cockroaches);

Allergens derived from fungi (e.g. Alternaria, Aspergillus, Botulinum, Candida, Cephalosporium, Carbaria, Epicoccum, Epidermis, Fusarium, Helminthosporumium, Chain Cladosporium, Mucoraceae, Peniculium, Pullularia pullulans, or Rhizopus);

Allergens derived from animal hair (e.g. hair of dogs, cats, or birds);

Allergens derived from house dust;

Allergens derived from foods; and

Haptens involved in metal allergy.

Examples of the diseases affected by the infectious pathogen include:

Viral diseases such as those affected by infections from Adenovirus, Herpes virus (e.g. HSV-I, HSV-II, CMV, or VZV), Poxvirus (e.g. pressure ulcer or vaccinia, or orthopox virus such as contagious molluscum), Picornavirus (e.g. rhinovirus or enterovirus), Orthomyxovirus (e.g. influenza virus), Paramyxovirus (e.g. parainfluenza virus, mumps virus, measles virus, or respiratory syncytial virus [RSV]), Coronavirus (e.g. SARS), Papovavirus (e.g. papilloma viruses such as the ones that cause genital warts, vulgaris, or plantar costus), Hepadnavirus (e.g. hepatitis B virus), Flavivirus (e.g. hepatitis C virus or dengue virus), or Retroviruses (e.g.lentivirus such as HIV);

Bacterial diseases such as those affected by infections from Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococci, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

Fungal diseases such as Chlamydia, Candidiasis, Aspergillosis, Histoplasmosis, or Cryptococcal meningitis; and

Other diseases such as Malaria, Pneumocystis carinii pneumonia, Leishmaniasis, Cryptosporidiosis, Toxoplasmosis, or Trypanosoma infection.

Particularly suitable antigens are Ovalbumin (OVA), Pneumococci, Influenza vaccines, Cryj1 (a major allergen of cedar pollen), or HPV16 recombinant protein.

Only one type of antigen may be used, or two or more types thereof may be used in combination. The content of the antigen in the vaccine composition is, for example, in the range of 1×10⁻⁷ to 1×10⁻¹ mass %, preferably in the range of 1×10⁻⁶ to 1×10⁻² mass %, more preferably in the range of 2×10⁻⁶ to 2×10⁻³ mass %.

As described above, the MOF is formed with a combination of metal(s) and multidentate ligand(s). The mechanism by which the MOF acts as adjuvant is not perfectly clear. The inventors however have attributed the reason to the metal and/or ligand in the MOF interacting with antigens and/or immune cells in some ways. As used herein, the “multidentate ligand” means a ligand that can form two or more coordinate bond.

Any kinds of MOFs can be used in the vaccine composition. Appropriately combining the type and coordination number of the metal ion with the type and topology of the multidentate ligand leads to a MOF with a desired structure. The MOF may be configured to decompose in vivo. The decomposition would expose the metal and the ligand constituting the MOF, by which the MOF might function as adjuvant more efficiently. The MOF can be crystalline or amorphous.

The metal elements in the MOF can be, for example, any elements belonging to alkali metals (Group 1), alkaline earth metals (Group 2), or transition metals (Groups 3 to 12). From the viewpoint of biocompatibility, it is preferable to use at least one metal element selected from the group consisting of calcium, magnesium, iron, zinc, aluminum, potassium, and sodium. However, any metal elements other than these preferable elements can also be used as long as biocompatibility of a MOF as a whole is ensured.

The multidentate ligand in the MOF typically is an organic ligand, examples of which include carboxylate anion and heterocyclic compound. Examples of the carboxylic acid anion include dicarboxylic acid anion and tricarboxylic acid anion. Specific examples include anions of citric acid, malic acid, terephthalic acid, isophthalic acid, trimesic acid, and derivatives thereof. Examples of the heterocyclic compound include bipyridine, imidazole, adenine, and derivatives thereof. Alternatively, the ligand may be an amine compound, a sulfonate anion, or a phosphate anion. The MOF may further contain monodentate ligand(s).

The combination of the metal and the ligand forming the MOF can be appropriately determined according to the expected function and the desired pore size. The MOF may contain two or more types of metal elements, and may contain two or more types of ligands. The MOF can be surface-modified with a polymer or other modifiers.

Specific examples of the MOF include those listed in Table 1 of the Non-Patent Literature 2. Those shown in Tables 1 to 3 below may also be used as the MOF. These are non-limiting lists, and other MOFs can also be used.

TABLE 1

| Name/ | Metal | |
|---|---|---|
| Abbreviation | (Cation) | Ligand (Anion) |
| CPL-1 | Cu | pzdc (2,3-pyrazinedicarboxylic acid), |
| | | pyz (pyrazine) |
| Cu₃(btc)₂ | Cu | BTC (trimesic acid) |
| Zn₂(14bdc)₂(dabco) | Zn | BDC (terephthalic acid), dabco (1,4- |
| | | diazabicyclo[2,2,2]octane) |
| ZIF-8 | Zn | imidazole |
| HKUST-1 | Cu | 1,3,5-benzenetricarboxylic acid |
| Mg₃(C₁₂O₁₄H₁₀) | Mg | citric acid |
| Ca₂(C₈O₁₂H₆) | Ca | malic acid |
| Ca₃(C₁₂O₁₄H₁₀) | Ca | citric acid |
| Ca(C₄O₆H₄) | Ca | malic acid |
| Cu(IPA) | Cu | isophthalic acid |
| MgBDC-1 | Mg | BDC (terephthalic acid) |
| MgDHBDC-1 | Mg | DHBDC (2,5-dihydroxyterephthalic |
| | | acid) |
| MgOBA-1 | Mg | OBA (4,4′-oxobisbenzoic acid) |
| MgBTC-1 | Mg | BTC (trimesic acid) |
| MgBTB-1 | Mg | BTB (1,3,5-tri(4′-carboxy-4,4′- |
| | | biphenyl)benzene) |
| Mg BTB-2 | Mg | BTB (1,3,5-tri(4′-carboxy-4,4′- |
| | | biphenyl)benzene) |
| MgBTB-3 | Mg | BTB (1,3,5-tri(4′-carboxy-4,4′- |
| | | biphenyl)benzene) |
| MgBTB-4 | Mg | BTB (1,3,5-tri(4′-carboxy-4,4′- |
| | | biphenyl)benzene) |
| MgBBC-1 | Mg | BBC (4,4′-4″-benzene-1,3,5-triyl- |
| | | tri-biphenylcarboxylic acid) |
| MIL-100(Fe) | Fe | BTC (trimesic acid) |
| MIL-101 | Fe | BDC (terephthalic acid) |
| MIL-53 | Fe | BDC (terephthalic acid) |
| BioMIL-5 | Zn | azelaic acid |
| CaZol nMOF | Ca | zoledronic acid |
| IRMOF-2 | Zn | o-Br-BDC (o-bromoterephthalic acid) |
| IRMOF-3 | Zn | H₂N-BDC (2-aminoterephthalic acid) |
| IRMOF-4 | Zn | [C₃H₇O]₂-BDC |
| IRMOF-5 | Zn | [C₅H₁₁O]₂-BDC |
| IRMOF-6 | Zn | [C₂H₄]-BDC |
| IRMOF-7 | Zn | 1,4-NDC (1,4-naphthalenedicarboxylic |
| | | acid) |
| IRMOF-8 | Zn | 2,6-NDC (2,6-naphthalenedicarboxylic |
| | | acid) |
| IRMOF-9 | Zn | BPDC (4,4′-biphenyldicarboxylic acid) |
| IRMOF-10 | Zn | BPDC (4,4′-biphenyldicarboxylic acid) |
| IRMOF-11 | Zn | HPDC (tetrahydropyrene-2,7-dicarboxylic |
| | | acid) |
| IRMOF-12 | Zn | HPDC (tetrahydropyrene-2,7-dicarboxylic |
| | | acid) |
| IRMOF-13 | Zn | PDC (pyrene dicarboxylic acid) |
| IRMOF-14 | Zn | PDC (pyrene dicarboxylic acid) |
| IRMOF-15 | Zn | TPDC (terphenyl dicarboxylic acid) |
| IRMOF-16 | Zn | TPDC (terphenyl dicarboxylic acid) |

TABLE 2

| Name/ | Metal | |
|---|---|---|
| Abbreviation | (Cation) | Ligand (Anion) |
| Zn₃(BTC)₂ | Zn | BTC (trimesic acid) |
| Zn₄O(NDC) | Zn | 1,4-NDC (1,4-naphthalenedicarboxylic acid) |
| Mg(Formate) | Mg | formic acid |
| Fe(Formate) | Fe | formic acid |
| Mg(C₆H₄O₆) | Mg | DHBDC (2,5-dihydroxyterephthalic acid) |
| ZnC₂H₄BDC | Zn | [C₂H₄]-BDC |
| MOF-49 | Zn | m-BDC |
| BPR95A2 | Zn | BDC (terephthalic acid) |
| BPR76D5 | Zn | BzPDC |
| BPR68D10 | Zn | BTC (trimesic acid) |
| BPR56E1 | Zn | BDC (terephthalic acid) |
| BPR49B1 | Zn | BDC (terephthalic acid) |
| BPR43G2 | Zn | BDC (terephthalic acid) |
| NO336 | Fe | formic acid |
| NO335 | Fe | formic acid |
| NO333 | Fe | formic acid |
| PCN-14 | Nb | 5,5′-(9,10-anthracenediyl) diisophosphate |
| Zn₄BNDC | Zn | BNDC (1,1′-binaphthyl-4,4′-dicarboxylic acid) |
| Zn₃(BPDC) | Zn | BPDC (4,4′-biphenyldicarboxylic acid) |
| ZnDBP | Zn | DBP (dibenzyl phosphate) |
| Zn₃(PDC)_{2.5} | Zn | PDC (pyrene dicarboxylic acid) |
| Zn(HPDC) | Zn | HPDC (tetrahydropyrene-2,7-dicarboxylic acid) |
| Zn(NDC) | Zn | 2,6-NDC (2,6-naphthalenedicarboxylic acid) |
| MOF-37 | Zn | 2,6-NDC (2,6-naphthalenedicarboxylic acid) |
| MOF-20 | Zn | 2,6-NDC (2,6-naphthalenedicarboxylic acid) |
| MOF-12 | Zn | ATC (1,3,5,7-adamantanetetracarboxylic acid) |
| Zn(ADC) | Zn | ADC (acetylenedicarboxylic acid) |
| MOF-0 | Zn | BTC (trimesic acid) |
| MOF-2 | Zn | BDC (terephthalic acid) |
| MOF-3 | Zn | BDC (terephthalic acid) |
| MOF-4 | Zn | BTC (trimesic acid) |
| MOF-5 | Zn | BDC (terephthalic acid) |
| MOF-38 | Zn | BTC (trimesic acid) |
| MOF-31 | Zn | ADC (acetylenedicarboxylic acid) |
| MOF-69A | Zn | BPDC (4,4′-biphenyldicarboxylic acid) |
| MOF-69B | Zn | 2,6-NDC (2,6-naphthalenedicarboxylic acid) |
| MOF-33 | Zn | ATB (adamantanetetrabenzoic acid) |
| MOF-36 | Zn | MTB (methanetetrabenzoic acid) |
| MOF-39 | Zn | BTB (1,3,5-tri(4′-carboxy-4,4′-biphenyl)benzene) |

TABLE 3

| | Metal | |
|---|---|---|
| Name/ | (Cat- | |
| Abbreviation | ion) | Ligand (Anion) |
| NO305 | Fe | formic acid |
| NO306A | Fe | formic acid |
| BPR48A2 | Zn | BDC (terephthalic acid) |
| Zn(C₂O₄) | Zn | oxalic acid |
| MOF-48 | Zn | 2,6-NDC (2,6-naphthalenedicarboxylic |
| | | acid) |
| MOF-47 | Zn | BDC(CH₃)₄ |
| Zn₃(BTC)₂ | Zn | BTC (trimesic acid) |
| MOF-n | Zn | BTC (trimesic acid) |
| Zehex | Zn | BTB (1,3,5-tri(4′-carboxy-4,4′- |
| | | biphenyl)benzene) |
| AS16 | Fe | BDC (terephthalic acid) |
| AS27-3 | Fe | BDC (terephthalic acid) |
| AS54-3 | Fe | BPDC (4,4′-biphenyldicarboxylic acid) |
| AS61-4 | Fe | m-BDC |
| AS68-7 | Fe | m-BDC |
| Zn₈(ad)₄(PDAC)₆(OH)₂ | Zn | adenine, PDAC (1,4-diphenyl diacrylic |
| | | acid) |
| Zn₈(ad)₄(SBDC)₆(OH)₂ | Zn | adenine, SBDC (4,4′-stilbene |
| | | dicarboxylic acid) |
| Zn₈(ad)₄(BPDC)₆(OH)₂ | Zn | adenine, BPDC |
| Zn₈(ad)₄(NDC)₆(OH)₂ | Zn | adenine, 2,6-NDC |
| M-CPO-27 | Mg | DHBDC (2,5-dihydroxyterephthalic |
| | | acid) |
| bio-MOF-1 | Zn | adenine, BPDC |
| UMCM-1 | Zn | BTB (1,3,5-tri(4′-carboxy-4,4′- |
| | | biphenyl)benzene) |
| UMCM-2 | Zn | BTB (1,3,5-tri(4′-carboxy-4,4′- |
| | | biphenyl)benzene) |
| MOF-210 | Zn | BTE (4,4′,4″-[benzene-1,3,5-triyl-tris |
| | | (ethyne-2,1-diyl)] tribenzoic acid), |
| | | BPDC |
| bio-MOF-100 | Zn | adenine, BPDC |
| NU-110E | Cu | J. Am. Chem. Soc. 2012, 134, |
| | | 15016-15021 |
| CD-MOF-1 | K | γ-CD (γ-cyclodextrin) |
| porph@MOM-4 | Fe | porphyrin, BTC |
| porph@MOM-8 | Mg | porphyrin, BTC |
| porph@MOM-9 | Zn | porphyrin, BTC |
| ZnPO-MOF | Zn | metalloporphyrin pyridyl, TCPB |
| | | (1,2,4,5-Tetrakis(4- |
| | | carboxyphenyl)benzene) |
| Uio-66 | Fe | DCBDT (1,4-dicarboxylbenzene-2,3- |
| | | dithiolate) |
| Mg(H₂gal) | Mg | caustic acid (3,4,5-trihydroxybenzoic |
| | | acid) |

Particularly preferable MOFs include the followings.

TABLE 4

| Abbreviation | Metal | Ligand |
|---|---|---|
| AP008 ZIF-8 | Zn²⁺ | 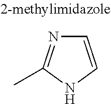 |
| AP004 MIL-100(Fe) | Fe³⁺ | 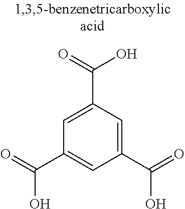 [SMILES: O=C(O)c1cc(C(=O)O)cc(C(=O)O)c1] |
| AP006 Al(Fumarate) | Al³⁺ | 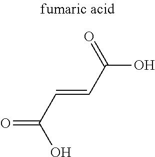 |
| AP005 MIL-53(Al) | Al³⁺ | 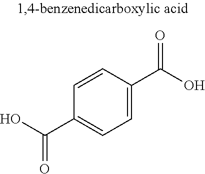 [SMILES: O=C(O)c1ccc(C(=O)O)cc1] |

TABLE 5

| Abbreviation | Metal | Ligand |
|---|---|---|
| AP101 | Ca²⁺ | 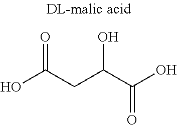 |
| AP104 BioMIL-3 | Ca²⁺ | 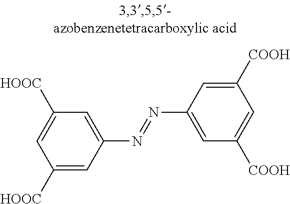 [SMILES: O=C(O)c1cc(/N=N/c2cc(C(=O)O)cc(C(=O)O)c2)cc(C(=O)O)c1] |
| AP009 Mg(Formate) | Mg²⁺ | 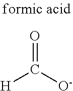 |
| AP014 | La³⁺ | 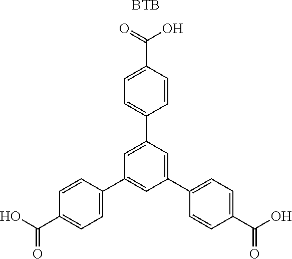 |

TABLE 6

| Abbreviation | Metal | Ligand |
|---|---|---|
| AP102 | Ca²⁺ | 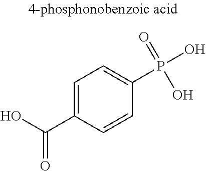 [SMILES: O=C(O)c1ccc(P(=O)(O)O)cc1] |
| AP103 | Ca²⁺ | 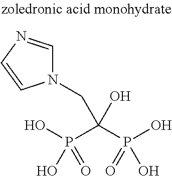 [SMILES: O=P(O)(O)C(O)(Cn1ccnc1)P(=O)(O)O] |
| AP105 | Ca²⁺ | 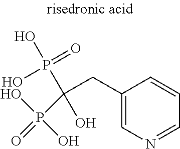 [SMILES: O=P(O)(O)C(O)(Cc1cccnc1)P(=O)(O)O] |

TABLE 7

| Abbreviation | Metal | Ligand |
|---|---|---|
| AP107 | Al³⁺ | 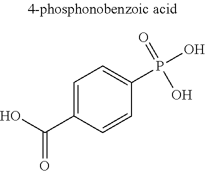 [SMILES: O=C(O)c1ccc(P(=O)(O)O)cc1] |
| AP106 | Mg²⁺ | 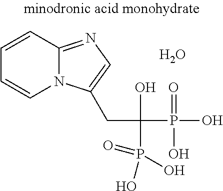 |
| AP108 | Ca²⁺ | 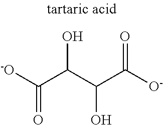 |
| AP015 | Ca²⁺ | 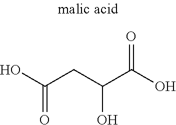 |

TABLE 8

| Abbreviation | Metal | Ligand |
|---|---|---|
| AP001 | Cu²⁺ | 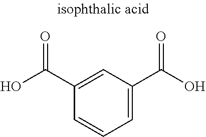 [SMILES: O=C(O)c1cccc(C(=O)O)c1] |
| AP003 Fe-BTC | Fe³⁺ | 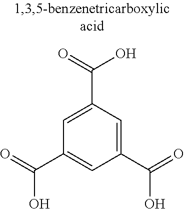 [SMILES: O=C(O)c1cc(C(=O)O)cc(C(=O)O)c1] |
| Ni-MOF-74 | Ni²⁺ | 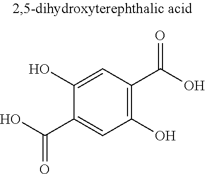 [SMILES: O=C(O)c1cc(O)c(C(=O)O)cc1O] |
| Co-MOF-74 | Co²⁺ | 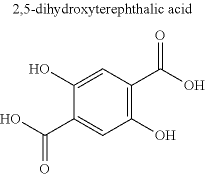 [SMILES: O=C(O)c1cc(O)c(C(=O)O)cc1O] |

TABLE 9

| Abbreviation | Metal | Ligand |
|---|---|---|
| MIL-88-A | Fe²⁺ | 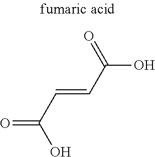 |
| MIL-88-B | Fe²⁺ | 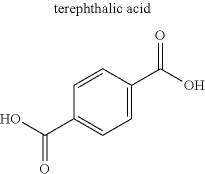 [SMILES: O=C(O)c1ccc(C(=O)O)cc1] |

Only one type of MOF may be used, or two or more types thereof may be used in combination. The content of the MOF in the vaccine composition is, for example, in the range of 1×10⁻⁷ to 99.9999999 mass %, preferably in the range of 1×10⁻⁶ to 99.999999 mass %, and more preferably in the range of 5×10⁻⁶ to 99.99999 mass %.

The vaccine composition according to one embodiment of the present invention may further contain an immune signal transducer. Adopting such a configuration can further enhance the effect of administering the vaccine composition. As used herein, the “immune signal transducer” means any substance used for transmitting an immune signal for inducing activation and/or differentiation of immune cells. The immune signal transducer may be, for example, cytokines such as interleukins, chemokines, interferons, hematopoietic factors, cell growth factors, or cell necrosis factors, or may be small molecules such as gas molecules that will be described later. As used herein, the “small molecule” means a molecule having a molecular weight of 1000 or less.

The immune signal transducer is, for example, a factor that is configured to act on lymphocytes (T cells, B cells, NK cells, etc.), monocytes (macrophages, Langerhans cells, dendritic cells, etc.), granulocytes (neutrophils, eosinophils, basophils, etc.) and/or keratinocytes. The immune signal transducer is, for example, a factor that is configured to induce differentiation of helper T cells, which are a type of lymphocyte, into various lineages such as Th1 cells, Th2 cells, Treg cells, Th17 cells, Tfh cells, or memory T cells. When the immune signal transducer induces Th1 cells, the vaccine composition according to the present invention can be used, for example, for cancer vaccines or infectious disease vaccines. When the immune signal transducer induces Th2 cells, the vaccine composition according to the present invention can be used, for example, for infectious disease vaccines or lifestyle-related disease vaccines. When the immune signal transducer induces Treg cells, the vaccine composition according to the present invention can be used, for example, for allergy vaccines. When the immune signal transducer induces Th17 cells, the vaccine composition according to the present invention can be used, for example, for infectious disease vaccines. When the immune signal transducer induces Tfh cells, the vaccine composition according to the present invention can be used, for example, for infectious disease vaccines. When the immune signal transducer induces memory T cells, the vaccine composition according to the present invention can be used, for example, for infectious disease vaccines or cancer vaccines.

It is preferable that at least a part of the immune signal transducer is contained in the pores of the MOF. This allows for more stable and quantitative administration of the immune signal transducer. In such a case, the other part of the immune signal transducer may be attached to the surface of the antigen and/or the MOF. Alternatively, most of the immune signal transducer may be contained in the pores of the MOF.

When at least a part of the immune signal transducer is contained in the pores of the MOF, it is preferable that the MOF has an irreversible adsorption/desorption profile. That is, the MOF preferably retains a larger amount of guest molecules at the time of desorption than the amount of guest molecules at the time of adsorption at the same pressure. It is particularly preferable that the residual amount of the guest molecules in the MOF is non-zero after performing the adsorption process from a vacuum state to a pressurized state and then performing the desorption process from the pressurized state to the vacuum state. This enables easier retention of the immune signal transducer in the pores of the MOF under the condition of low pressure (e.g. at atmospheric pressure).

When at least a part of the immune signal transducer is contained in the pores of the MOF, it is also preferable that the MOF is configured to decompose in vivo to release at least a part of the immune signal transducer. This allows finer adjustment of the dose and the release rate of the immune signal transducer. The decomposition may also induce more exposure of the metal and the ligand of the MOF, thereby further enhancing the function of the MOF as an adjuvant.

As described above, the immune signal transducer can be a small molecule. This makes it easier to include at least a part of the immune signal transducer in the pores of the MOF. As used herein, again, the “small molecule” means a molecule having a molecular weight of 1000 or less.

More preferably, the immune signal transducer is a gas under the condition of 25° C. and 100 kPa (i.e. SATP). This makes it still easier to include at least a part of the immune signal transducer in the pores of the MOF.

In recent years, it has been becoming clear that small molecules such as gas molecules function as immune signal transducers. For example, gas molecules such as nitric oxide, carbon monoxide, carbon dioxide, hydrogen sulfide, or methane have been shown to act on immunocompetent cells. However, there have been no method for stably and quantitatively administering small molecules such as gas molecules into a living body, and a person skilled in the art has not tried it yet because of its anticipated difficulty. The present inventors have however found that small molecules such as gas molecules can be stably and quantitatively administered in vivo by using small molecules such as gas molecules along with the MOF.

There are no particular limitations on the small molecules or gas molecules used as immune signal transducers. Examples of such an immune signal transducer include compounds shown in Table 10 below. These are non-limiting lists, and other small molecules or gas molecules may be used.

TABLE 10

| | |
|---|---|
| Diatomic molecules | Nitrogen, oxygen, hydrogen, fluorine, chlorine, |
| | bromine, iodine |
| Noble gases | Helium, neon, argon, krypton, xenon, radon |
| Carbon oxides | Carbon monoxide, carbon dioxide |
| Nitrogen compounds | Ammonia, nitric oxide, nitrogen dioxide, |
| | dinitrogen monoxide, dinitrogen tetroxide, |
| | dinitrogen trioxide, dinitrogen pentoxide, |
| | dimethylamine, trimethylamine |
| Sulfur compounds | Sulfur dioxide, hydrogen sulfide, methanethiol, |
| | dimethyl sulfide |
| Alkanes | Methane, ethane, propane, butane, |
| | halogenated methane |
| Alkenes | Ethylene, propylene, butadiene |
| Alkynes | Acetylene |
| Alcohols | Methanol, ethanol, propanol |
| Aldehydes | Formaldehyde, acetaldehyde |
| Carboxylic acids | Formic acid, acetic acid, citric acid, malic acid |
| Ethers | Dimethyl ether, diethyl ether |
| Aromatic compounds | Benzene, toluene |
| Others | Water, bioactive substances |

Only one type of immune signal transducer may be used, or two or more types thereof may be used in combination. The content of the immune signal transducer in the vaccine composition is, for example, in the range of 1×10⁻⁷ to 40% by mass, preferably in the range of 1×10⁻⁶ to 30% by mass, and more preferably in the range of 5×10⁻⁵ to 25 mass %.

Any methods can be used for introducing the immune signal transducer into the pores of the MOF. For example, a solution or dispersion of a MOF may be mixed with a solution or dispersion of an immune signal transducer. Alternatively, a solid MOF may be exposed to an immune signal transducer or a solution or dispersion thereof. When the immune signal transducer is a gas, the MOF may be simply exposed to the gas.

The vaccine composition according to one embodiment of the present invention may further contain other adjuvant(s) than the MOF. The vaccine composition may also contain immunostimulant(s) such as a TLR ligand, an RLR ligand, an NLR ligand, or a cyclic dinucleotide.

The vaccine composition according to one embodiment of the present invention can be dissolved or dispersed in a solvent when in use. Examples of such solvents include physiological saline, phosphate buffered saline (PBS), glycerin, propylene glycol, polyethylene glycol, fats, or oils.

The vaccine composition according to the present invention can be administered to a subject by any method. As used herein, the “subject” refers to any animal whose immune response can be induced upon administration of vaccine composition in the practical stage. The animal typically is a mammal including humans, such as mice, rats, dogs, cats, rabbits, horses, cow, sheep, pig, goat, monkey, chimpanzee, ferret, mole, etc. A particularly preferred subject is a human.

The vaccine composition according to one embodiment of the present invention may be configured to be administered, for example, on a skin and/or mucous membrane of a subject.

In the case of transdermal administration, the vaccine composition may be any formulation commonly used for transdermal administration. For example, liquid for external use such as liniments or lotions, external sprays such as aerosols, ointments, plasters, creams, gels, or patches such as tapes or poultices can be used. The classification, definition, properties, and production method of these compositions are well known in the art, and can be found, for example, in the Japanese Pharmacopoeia 16th edition.

In the case of mucosal administration, the vaccine composition may be any formulation commonly used for mucosal administration such as sublingual, nasal, buccal, rectal or vaginal administration. For example, semi-solid preparations such as gel (jelly), cream, ointment, or plasters, liquid preparations, solid preparations such as powders, fine granules, granules, films, tablets, or orally disintegrating tablets, sprays for mucous membranes such as aerosols, or inhalants can be used. The classification, definition, properties, and production method of these compositions are well known in the art, and can be found, for example, in the Japanese Pharmacopoeia 16th edition.

The vaccine composition according to one embodiment of the present invention can also be configured to be administered, for example, by intradermal injection, subcutaneous injection, or intramuscular injection. In the case of intradermal, subcutaneous, or intramuscular administration, the composition may be in a form that has a certain fluidity that can be administered by injection, such as a liquid, suspension, cream, and the like. The classification, definition, properties, and production method of these compositions are well known in the art, and can be found, for example, in the Japanese Pharmacopoeia 16th edition.

The vaccine composition may further contain additive(s) if necessary. The additives can be selected depending, for example, upon main component of the base, compatibility with the antigen and/or the MOF, or the intended dosage regimen. Examples of the additives include skin permeability enhancers, isotonic agents, antiseptic/disinfectants, antioxidants, solubilizers, solubilizing agents, suspending agents, fillers, pH adjusters, stabilizers, absorption enhancers, release rate controllers, colorants, plasticizers, adhesives, or their combinations.

The adjuvant according to the present disclosure includes a MOF. This adjuvant may be used independently from the antigen. For example, the adjuvant may be administered separately after the antigen is administered to a subject. Alternatively, the antigen may be administered after the adjuvant is administered.

The MOF as the adjuvant may be configured to decompose in vivo. The MOF may further contain an immune signal transducer in its pores. The MOF may also be configured to decompose in the living body to release at least a part of the immune signal transducer contained in the pores. Similar methods as explained above can be used for introducing at least a part of the immune signal transducer in the pores of the MOF. Also, similar administration methods as explained above in regard to the vaccine composition can be used for administering the adjuvant.

As described above, the immune signal transducer is, for example, a factor that is configured to induce activation and/or differentiation of lymphocytes (T cells, B cells, NK cells, etc.), monocytes (macrophages, Langerhans cells, dendritic cells, etc.), granulocytes (neutrophils, eosinophils, basophils, etc.) and/or keratinocytes. The immune signal transducer is, for example, a factor that is configured to induce differentiation of naive helper T cells into various lineages such as Th1 cells, Th2 cells, Treg cells, Th17 cells, Tfh cells, or memory T cells. When the immune signal transducer induces Th1 cells, the adjuvant according to the present invention can be used, for example, for cancer vaccines, infectious disease vaccines, or as concomitant drugs with anti-cancer agents. When the immune signal transducer induces Th2 cells, the adjuvant according to the present invention can be used, for example, for infectious disease vaccines or lifestyle-related disease vaccines. When the immune signal transducer induces Treg cells, the adjuvant according to the present invention can be used, for example, for allergy vaccines or organ transplantations. When the immune signal transducer induces Th17 cells, the adjuvant according to the present invention can be used, for example, for infectious disease vaccines. When the immune signal transducer induces Tfh cells, the adjuvant according to the present invention can be used, for example, for infectious disease vaccines. When the immune signal transducer induces memory T cells, the adjuvant according to the present invention can be used, for example, for infectious disease vaccines or cancer vaccines.

### EXAMPLES

[Preparation of Sample Solutions]

### Example 1

NO (nitrogen monoxide, Kyoto Teijin) was bubbled into 100 mL of physiological saline (Otsuka Normal Saline, Otsuka Pharmaceutical) at room temperature for 6 hours to prepare NO-saturated physiological saline. To 10 mL of the obtained solution was added 1 mg of ZIF-8 (Basolite Z1200, Sigma-Aldrich) and 1 mg of OVA (egg-derived albumin, Wako), and these were mixed to provide a sample solution.

### Example 2

Another sample solution was prepared by adding 1 mg of ZIF-8 (Basolite Z1200, Sigma-Aldrich) and 1 mg of OVA (egg-derived albumin, Wako) to 10 mL of physiological saline (Otsuka Normal Saline, Otsuka Pharmaceutical).

### Comparative Example 1

Physiological saline (Otsuka Normal Saline, Otsuka Pharmaceutical) itself was used as a sample solution.

### Comparative Example 2

1 mg of OVA (egg-derived albumin, Wako) was added to and mixed with 10 mL of physiological saline (Otsuka Normal Saline, Otsuka Pharmaceutical) to obtain a sample solution.

### Reference Example 1

1 mg of ZIF-8 (Basolite Z1200, Sigma-Aldrich) was added to and mixed with 10 mL of physiological saline (Otsuka Normal Saline, Otsuka Pharmaceutical) to obtain a sample solution.

### Reference Example 2

NO (nitrogen monoxide, Kyoto Teijin) was bubbled in 100 mL of physiological saline (Otsuka Normal Saline, Otsuka Pharmaceutical) at room temperature for 6 hours to prepare NO saturated physiological saline. To 10 mL of the obtained solution was added 1 mg of ZIF-8 (Basolite Z1200, Sigma-Aldrich), and these were mixed to provide a sample solution.

The above configuration is summarized in Table 11 below.

TABLE 11

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Comp. Ex. 1 | — | — | — | — | Physiological saline | 100 | — | — |
| Ref. Ex. 1 | — | — | ZIF-8 | 100 | Physiological saline | 100 | — | — |
| Ref. Ex. 2 | — | — | ZIF-8 | 100 | Physiological saline | 100 | NO | 1.8 |
| Example 1 | OVA | 100 | ZIF-8 | 100 | Physiological saline | 100 | NO | 1.8 |
| Example 2 | OVA | 100 | ZIF-8 | 100 | Physiological saline | 100 | — | — |
| Comp. Ex. 2 | OVA | 100 | — | — | Physiological saline | 100 | — | — |

### Examples 3 to 6

Sample solutions were prepared in the same manner as in Example 1 except that the antigens shown in Table 12 below were used instead of OVA.

TABLE 12

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Example 1 | OVA | — | ZIF-8 | 100 | Physiological saline | 100 | NO | 1.8 |
| Example 3 | Pneumococcus | — | ZIF-8 | 100 | Physiological saline | 100 | NO | 1.8 |
| Example 4 | Influenza vaccine | — | ZIF-8 | 100 | Physiological saline | 100 | NO | 1.8 |
| Example 5 | Cyj1 | 100 | ZIF-8 | 100 | Physiological saline | 100 | NO | 1.8 |
| Example 6 | HPV16 recombinant protein | 100 | ZIF-8 | 100 | Physiological saline | 100 | NO | 1.8 |

### Examples 7 to 35

Sample solutions were prepared in the same manner as in Example 1 except that the substances shown in Table 13 below were used instead of NO as immune signal transducers.

TABLE 13

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Example 1 | OVA | 100 | ZIF-8 | 100 | Physiological saline | 100 | NO | Saturated |
| Example 7 | OVA | 100 | ZIF-8 | 100 | Physiological saline | 100 | CO | Saturated |
| Example 8 | OVA | 100 | ZIF-8 | 100 | Physiological saline | 100 | CO₂ | Saturated |
| Example 9 | OVA | 100 | ZIF-8 | 100 | Physiological saline | 100 | N₂ | Saturated |
| Example 10 | OVA | 100 | ZIF-8 | 100 | Physiological saline | 100 | O₂ | Saturated |
| Example 11 | OVA | 100 | ZIF-8 | 100 | Physiological saline | 100 | H₂ | Saturated |
| Example 12 | OVA | 100 | ZIF-8 | 100 | Physiological saline | 100 | H₂S | Saturated |
| Example 13 | OVA | 100 | ZIF-8 | 100 | Physiological saline | 100 | S₂O | Saturated |
| Example 14 | OVA | 100 | ZIF-8 | 100 | Physiological saline | 100 | CH₄ | Saturated |
| Example 15 | OVA | 100 | ZIF-8 | 100 | Physiological saline | 100 | C₂H₆ | Saturated |
| Example 16 | OVA | 100 | ZIF-8 | 100 | Physiological saline | 100 | C₃H₈ | Saturated |
| Example 17 | OVA | 100 | ZIF-8 | 100 | Physiological saline | 100 | C₄H₁₀ | Saturated |
| Example 18 | OVA | 100 | ZIF-8 | 100 | Physiological saline | 100 | C₂H₄ | Saturated |
| Example 19 | OVA | 100 | ZIF-8 | 100 | Physiological saline | 100 | C₃H₆ | Saturated |
| Example 20 | OVA | 100 | ZIF-8 | 100 | Physiological saline | 100 | C₂H₄ | Saturated |
| Example 21 | OVA | 100 | ZIF-8 | 100 | Physiological saline | 100 | CH₃NH₂ | Saturated |
| Example 22 | OVA | 100 | ZIF-8 | 100 | Physiological saline | 100 | (CH₃)₂NH | Saturated |
| Example 23 | OVA | 100 | ZIF-8 | 100 | Physiological saline | 100 | NH₃ | Saturated |
| Example 24 | OVA | 100 | ZIF-8 | 100 | Physiological saline | 100 | CH₃SH | Saturated |
| Example 25 | OVA | 100 | ZIF-8 | 100 | Physiological saline | 100 | (CH₃)₃N | Saturated |
| Example 26 | OVA | 100 | ZIF-8 | 100 | Physiological saline | 100 | CH₃Cl | Saturated |
| Example 27 | OVA | 100 | ZIF-8 | 100 | Physiological saline | 100 | CH₃Br | Saturated |
| Example 28 | OVA | 100 | ZIF-8 | 100 | Physiological saline | 100 | He | Saturated |
| Example 29 | OVA | 100 | ZIF-8 | 100 | Physiological saline | 100 | F₂ | Saturated |
| Example 30 | OVA | 100 | ZIF-8 | 100 | Physiological saline | 100 | Ne | Saturated |
| Example 31 | OVA | 100 | ZIF-8 | 100 | Physiological saline | 100 | Cl₂ | Saturated |
| Example 32 | OVA | 100 | ZIF-8 | 100 | Physiological saline | 100 | Ar | Saturated |
| Example 33 | OVA | 100 | ZIF-8 | 100 | Physiological saline | 100 | Kr | Saturated |
| Example 34 | OVA | 100 | ZIF-8 | 100 | Physiological saline | 100 | Xe | Saturated |
| Example 35 | OVA | 100 | ZIF-8 | 100 | Physiological saline | 100 | Rn | Saturated |

### Examples 36 to 145

Sample solutions were prepared in the same manner as in Example 1 except that the substances shown in Table 14 to 16 below were used instead of ZIF-8 as MOFs (i.e. as adjuvants). Abbreviations in Tables 14 to 16 are the same as those described in Tables 1 to 3, respectively.

TABLE 14

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Example 1 | OVA | 100 | ZIF-8 | 100 | Physiological saline | 100 | NO | Saturated |
| Example 36 | OVA | 100 | CPL-1 | 100 | Physiological saline | 100 | NO | Saturated |
| Example 37 | OVA | 100 | Cu₃(btc)₂ | 100 | Physiological saline | 100 | NO | Saturated |
| Example 38 | OVA | 100 | Zn₂(14bdc)₂(dabco) | 100 | Physiological saline | 100 | NO | Saturated |
| Example 39 | OVA | 100 | ZIF-8 | 100 | Physiological saline | 100 | NO | Saturated |
| Example 40 | OVA | 100 | HKUST-1 | 100 | Physiological saline | 100 | NO | Saturated |
| Example 41 | OVA | 100 | Mg₃(C₁₂O₁₄H₁₀) | 100 | Physiological saline | 100 | NO | Saturated |
| Example 42 | OVA | 100 | Ca₂(C₈O₁₂H₆) | 100 | Physiological saline | 100 | NO | Saturated |
| Example 43 | OVA | 100 | Ca₃(C₁₂O₁₄H₁₀) | 100 | Physiological saline | 100 | NO | Saturated |
| Example 44 | OVA | 100 | Ca(C₄O₆H₄) | 100 | Physiological saline | 100 | NO | Saturated |
| Example 45 | OVA | 100 | Cu(IPA) | 100 | Physiological saline | 100 | NO | Saturated |
| Example 46 | OVA | 100 | MgBDC-1 | 100 | Physiological saline | 100 | NO | Saturated |
| Example 47 | OVA | 100 | MgDHBDC-1 | 100 | Physiological saline | 100 | NO | Saturated |
| Example 48 | OVA | 100 | MgOBA-1 | 100 | Physiological saline | 100 | NO | Saturated |
| Example 49 | OVA | 100 | MgBTC-1 | 100 | Physiological saline | 100 | NO | Saturated |
| Example 50 | OVA | 100 | MgBTB-1 | 100 | Physiological saline | 100 | NO | Saturated |
| Example 51 | OVA | 100 | MgBTB-2 | 100 | Physiological saline | 100 | NO | Saturated |
| Example 52 | OVA | 100 | MgBTB-3 | 100 | Physiological saline | 100 | NO | Saturated |
| Example 53 | OVA | 100 | MgBTB-4 | 100 | Physiological saline | 100 | NO | Saturated |
| Example 54 | OVA | 100 | MgBBC-1 | 100 | Physiological saline | 100 | NO | Saturated |
| Example 55 | OVA | 100 | MIL-100(Fe) | 100 | Physiological saline | 100 | NO | Saturated |
| Example 56 | OVA | 100 | MIL-101 | 100 | Physiological saline | 100 | NO | Saturated |
| Example 57 | OVA | 100 | MIL-53 | 100 | Physiological saline | 100 | NO | Saturated |
| Example 58 | OVA | 100 | BioMIL-5 | 100 | Physiological saline | 100 | NO | Saturated |
| Example 59 | OVA | 100 | CaZol nMOF | 100 | Physiological saline | 100 | NO | Saturated |
| Example 60 | OVA | 100 | IRMOF-2 | 100 | Physiological saline | 100 | NO | Saturated |
| Example 61 | OVA | 100 | IRMOF-3 | 100 | Physiological saline | 100 | NO | Saturated |
| Example 62 | OVA | 100 | IRMOF-4 | 100 | Physiological saline | 100 | NO | Saturated |
| Example 63 | OVA | 100 | IRMOF-5 | 100 | Physiological saline | 100 | NO | Saturated |
| Example 64 | OVA | 100 | IRMOF-6 | 100 | Physiological saline | 100 | NO | Saturated |
| Example 65 | OVA | 100 | IRMOF-7 | 100 | Physiological saline | 100 | NO | Saturated |
| Example 66 | OVA | 100 | IRMOF-8 | 100 | Physiological saline | 100 | NO | Saturated |
| Example 67 | OVA | 100 | IRMOF-9 | 100 | Physiological saline | 100 | NO | Saturated |
| Example 68 | OVA | 100 | IRMOF-10 | 100 | Physiological saline | 100 | NO | Saturated |
| Example 69 | OVA | 100 | IRMOF-11 | 100 | Physiological saline | 100 | NO | Saturated |
| Example 70 | OVA | 100 | IRMOF-12 | 100 | Physiological saline | 100 | NO | Saturated |
| Example 71 | OVA | 100 | IRMOF-13 | 100 | Physiological saline | 100 | NO | Saturated |
| Example 72 | OVA | 100 | IRMOF-14 | 100 | Physiological saline | 100 | NO | Saturated |
| Example 73 | OVA | 100 | IRMOF-15 | 100 | Physiological saline | 100 | NO | Saturated |
| Example 74 | OVA | 100 | IRMOF-16 | 100 | Physiological saline | 100 | NO | Saturated |

TABLE 15

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Example 75 | OVA | 100 | Zn₃(BTC)₂ | 100 | Physiological saline | 100 | NO | Saturated |
| Example 76 | OVA | 100 | Zn₄O(NDC) | 100 | Physiological saline | 100 | NO | Saturated |
| Example 77 | OVA | 100 | Mg(Formate) | 100 | Physiological saline | 100 | NO | Saturated |
| Example 78 | OVA | 100 | Fe(Formate) | 100 | Physiological saline | 100 | NO | Saturated |
| Example 79 | OVA | 100 | Mg(C₆H₄O₆) | 100 | Physiological saline | 100 | NO | Saturated |
| Example 80 | OVA | 100 | ZnC₂H₄BDC | 100 | Physiological saline | 100 | NO | Saturated |
| Example 81 | OVA | 100 | MOF-49 | 100 | Physiological saline | 100 | NO | Saturated |
| Example 82 | OVA | 100 | BPR95A2 | 100 | Physiological saline | 100 | NO | Saturated |
| Example 83 | OVA | 100 | BPR76D5 | 100 | Physiological saline | 100 | NO | Saturated |
| Example 84 | OVA | 100 | BPR68D10 | 100 | Physiological saline | 100 | NO | Saturated |
| Example 85 | OVA | 100 | BPR56E1 | 100 | Physiological saline | 100 | NO | Saturated |
| Example 86 | OVA | 100 | BPR49B1 | 100 | Physiological saline | 100 | NO | Saturated |
| Example 87 | OVA | 100 | BPR43G2 | 100 | Physiological saline | 100 | NO | Saturated |
| Example 88 | OVA | 100 | NO336 | 100 | Physiological saline | 100 | NO | Saturated |
| Example 89 | OVA | 100 | NO335 | 100 | Physiological saline | 100 | NO | Saturated |
| Example 90 | OVA | 100 | NO333 | 100 | Physiological saline | 100 | NO | Saturated |
| Example 91 | OVA | 100 | PCN-14 | 100 | Physiological saline | 100 | NO | Saturated |
| Example 92 | OVA | 100 | Zn₄BNDC | 100 | Physiological saline | 100 | NO | Saturated |
| Example 93 | OVA | 100 | Zn₃(BPDC) | 100 | Physiological saline | 100 | NO | Saturated |
| Example 94 | OVA | 100 | ZnDBP | 100 | Physiological saline | 100 | NO | Saturated |
| Example 95 | OVA | 100 | Zn₃(PDC)_{2.5} | 100 | Physiological saline | 100 | NO | Saturated |
| Example 96 | OVA | 100 | Zn(HPDC) | 100 | Physiological saline | 100 | NO | Saturated |
| Example 97 | OVA | 100 | Zn(NDC) | 100 | Physiological saline | 100 | NO | Saturated |
| Example 98 | OVA | 100 | MOF-37 | 100 | Physiological saline | 100 | NO | Saturated |
| Example 99 | OVA | 100 | MOF-20 | 100 | Physiological saline | 100 | NO | Saturated |
| Example 100 | OVA | 100 | MOF-12 | 100 | Physiological saline | 100 | NO | Saturated |
| Example 101 | OVA | 100 | Zn(ADC) | 100 | Physiological saline | 100 | NO | Saturated |
| Example 102 | OVA | 100 | MOF-0 | 100 | Physiological saline | 100 | NO | Saturated |
| Example 103 | OVA | 100 | MOF-2 | 100 | Physiological saline | 100 | NO | Saturated |
| Example 104 | OVA | 100 | MOF-3 | 100 | Physiological saline | 100 | NO | Saturated |
| Example 105 | OVA | 100 | MOF-4 | 100 | Physiological saline | 100 | NO | Saturated |
| Example 106 | OVA | 100 | MOF-5 | 100 | Physiological saline | 100 | NO | Saturated |
| Example 107 | OVA | 100 | MOF-38 | 100 | Physiological saline | 100 | NO | Saturated |
| Example 108 | OVA | 100 | MOF-31 | 100 | Physiological saline | 100 | NO | Saturated |
| Example 109 | OVA | 100 | MOF-69A | 100 | Physiological saline | 100 | NO | Saturated |
| Example 110 | OVA | 100 | MOF-69B | 100 | Physiological saline | 100 | NO | Saturated |
| Example 111 | OVA | 100 | MOF-33 | 100 | Physiological saline | 100 | NO | Saturated |
| Example 112 | OVA | 100 | MOF-36 | 100 | Physiological saline | 100 | NO | Saturated |
| Example 113 | OVA | 100 | MOF-39 | 100 | Physiological saline | 100 | NO | Saturated |

TABLE 16

| | Antigen | | MOF | | Solvent | | Immune Signal Transducer | |
|---|---|---|---|---|---|---|---|---|
| | | Concentration | | Concentration | | Amount | | Concentration |
| | Name | [μg/mL] | Name | [μg/mL] | Name | [μL] | Name | [mM] |
| Example 114 | OVA | 100 | NO305 | 100 | Physiological saline | 100 | NO | Saturated |
| Example 115 | OVA | 100 | NO306A | 100 | Physiological saline | 100 | NO | Saturated |
| Example 116 | OVA | 100 | BPR48A2 | 100 | Physiological saline | 100 | NO | Saturated |
| Example 117 | OVA | 100 | Zn(C₂O₄) | 100 | Physiological saline | 100 | NO | Saturated |
| Example 118 | OVA | 100 | MOF-48 | 100 | Physiological saline | 100 | NO | Saturated |
| Example 119 | OVA | 100 | MOF-47 | 100 | Physiological saline | 100 | NO | Saturated |
| Example 120 | OVA | 100 | Zn₃(BTC)₂ | 100 | Physiological saline | 100 | NO | Saturated |
| Example 121 | OVA | 100 | MOF-n | 100 | Physiological saline | 100 | NO | Saturated |
| Example 122 | OVA | 100 | Zehex | 100 | Physiological saline | 100 | NO | Saturated |
| Example 123 | OVA | 100 | AS16 | 100 | Physiological saline | 100 | NO | Saturated |
| Example 124 | OVA | 100 | AS27-3 | 100 | Physiological saline | 100 | NO | Saturated |
| Example 125 | OVA | 100 | AS54-3 | 100 | Physiological saline | 100 | NO | Saturated |
| Example 126 | OVA | 100 | AS61-4 | 100 | Physiological saline | 100 | NO | Saturated |
| Example 127 | OVA | 100 | AS68-7 | 100 | Physiological saline | 100 | NO | Saturated |
| Example 128 | OVA | 100 | Zn₈(ad)₄(PDAC)₆(OH)₂ | 100 | Physiological saline | 100 | NO | Saturated |
| Example 129 | OVA | 100 | Zn₈(ad)₄(SBDC)₆(OH)₂ | 100 | Physiological saline | 100 | NO | Saturated |
| Example 130 | OVA | 100 | Zn₈(ad)₄(BPDC)₆(OH)₂ | 100 | Physiological saline | 100 | NO | Saturated |
| Example 131 | OVA | 100 | Zn₈(ad)₄(NDC)₆(OH)₂ | 100 | Physiological saline | 100 | NO | Saturated |
| Example 132 | OVA | 100 | M-CPO-27 | 100 | Physiological saline | 100 | NO | Saturated |
| Example 133 | OVA | 100 | bio-MOF-1 | 100 | Physiological saline | 100 | NO | Saturated |
| Example 134 | OVA | 100 | UMCM-1 | 100 | Physiological saline | 100 | NO | Saturated |
| Example 135 | OVA | 100 | UMCM-2 | 100 | Physiological saline | 100 | NO | Saturated |
| Example 136 | OVA | 100 | MOF-210 | 100 | Physiological saline | 100 | NO | Saturated |
| Example 137 | OVA | 100 | bio-MOF-100 | 100 | Physiological saline | 100 | NO | Saturated |
| Example 138 | OVA | 100 | NU-110E | 100 | Physiological saline | 100 | NO | Saturated |
| Example 139 | OVA | 100 | CD-MOF-1 | 100 | Physiological saline | 100 | NO | Saturated |
| Example 140 | OVA | 100 | porph@MOM-4 | 100 | Physiological saline | 100 | NO | Saturated |
| Example 141 | OVA | 100 | porph@MOM-8 | 100 | Physiological saline | 100 | NO | Saturated |
| Example 142 | OVA | 100 | porph@MOM-9 | 100 | Physiological saline | 100 | NO | Saturated |
| Example 143 | OVA | 100 | ZnPO-MOF | 100 | Physiological saline | 100 | NO | Saturated |
| Example 144 | OVA | 100 | Uio-66 | 100 | Physiological saline | 100 | NO | Saturated |
| Example 145 | OVA | 100 | Mg(H₂gal) | 100 | Physiological saline | 100 | NO | Saturated |

[Collection of Intraperitoneal Cells (PEC cells)]

A mouse was intraperitoneally administered with 2 mL of 4 wt % thioglycolic acid solution, and cells in its peritoneal cavity were taken out 3 days later. The collected cells were then washed with PBS (Phosphate Buffered Saline).

[Stimulation by Sample Solutions]

The PEC cells were dispensed in a 24-well plate at 1×10⁶ cells/well, and each sample was added thereto and incubated for 24 hours.

[Cytokine Measurement]

50 μL/well of the supernatant of the cell culture was used for an evaluation by an ELISA kit (Quantikine ELISA kit, R&D Systems) that corresponds to each cytokine (TNF-α, IL-6, IFN-γ, IL-12p40, IL-10) to be monitored. The results are summarized in Table 17 below.

TABLE 17

| | TNF-α | IL-6 | IL-10 | IL-12p40 | IFN-g |
|---|---|---|---|---|---|
| Comp. Ex. 1 | − | − | − | − | − |
| Ref. Ex. 1 | + | + | − | − | − |
| Ref. Ex. 2 | ++ | ++ | − | + | + |
| Example 1 | ++ | ++ | − | ++ | + |
| Example 2 | ++ | ++ | − | − | − |
| Comp. Ex. 2 | + | − | − | − | − |

(−): Less than twice the amount of cytokine released in Comparative Example 1
(+): Between twice and three times the amount of cytokine released in Comparative Example 1.
(++): Three or more times the amount of cytokine released in Comparative Example 1

[Measurement of OVA-Specific IgG Titer in Mouse Serum (ELISA Method)]

100 μL of OVA-containing solution (100 μg/mL) diluted with carbonate buffer was added to a 96-well plate for ELISA, and allowed to stand overnight. The wells were washed three times with a washing solution (PBS containing Tween 20), and 200 μL of a blocking solution obtained by diluting a blocking agent (Block Ace, Sumitomo Dainippon Pharma Co., Ltd.) with purified water to 4 g/100 mL was added and was left for 2 hours at room temperature. The wells were washed three times with the washing solution again.

Serum collected from a mouse in advance was centrifuged at 3000 g for 10 minutes at 4° C., and the obtained supernatant was collected. The above-mentioned supernatant was serially diluted to two times using a solution obtained by diluting the blocking agent with a phosphate buffer (Nacalai Tesque) to 0.4 g/100 mL, and 50 μL of the solution was added to each well and was left at room temperature for 2 hours.

The wells were washed three times with the washing solution, and an HRP-labeled anti-mouse IgG antibody (Goat-anti mouse IgG Fc HRP, BETHYL) was diluted to 10000 times with the solution obtained by diluting the blocking agent with a phosphate buffer (Nacalai Tesque) to 0.4 g/100 mL, and 100 μL of the solution was added to each well and was left at room temperature for 1 hour. The wells were washed three times with the washing solution, and 100 μL of TMB solution (ELISA POD TMB kit, Nacalai Tesque) was added, and was left in the dark for 30 minutes. 100 μL of 1M sulfuric acid solution was then added, and the absorbance at 450 nm was measured for the 96-well plate with a microplate reader (SpectraMax, Molecular Device). Based on the absorbance of the serially diluted samples, the IgG antibody titer in mouse serum was determined by Log 2 scale.

[Evaluation of Humoral Immunity Using Mice]

Using a liquid prepared as described above, a mouse immunity test was conducted using a model animal for humoral immunity evaluation. 200 μL of an injection sample was administered subcutaneously to the back of a mouse (BALB/c mouse, female 7 weeks old). One week after the administration, the same administration was again performed subcutaneously on the back of the mouse. Two weeks after the second administration, mouse serum was collected, and the serum OVA-specific IgG titer was measured by the ELISA method as described above.

[OVA Antigen-Specific CTL Measurement (ELISPOT Method)]

Splenocytes (3×10⁶ cells/well) and antigenic peptide (100 μM) or antigenic protein (100 μg/mL) were placed along with a culture solution in a well of an ELISPOT plate (R&D Systems) on which an anti-mouse IFN-γ antibody is immobilized. The cells were co-cultured at 37° C. under 5% CO₂ for 20 hours, and the number of IFN-γ producing cell spots (the number of spots per 3×10⁶ cells) was measured by ELISPOT method.

[Evaluation of Cellular Immunity Using Mice]

Using a liquid prepared as described above, a mouse immunity test was conducted using a model animal for cellular immunity evaluation. 200 μL of an injection was administered subcutaneously to the back of a mouse (C57BL/6 mouse, female 7 week old). One week after the administration, the same administration was again performed subcutaneously on the back of the mouse. One week after the second administration, mouse spleen was collected, and OVA antigen-specific CTL was measured by the ELISPOT method described above.

These results are summarized in Table 18 below.

TABLE 18

| | IgG | The number of CTLs |
|---|---|---|
| Comp. Ex. 1 | − | − |
| Ref. Ex. 1 | − | − |
| Ref. Ex. 2 | − | − |
| Example 1 | +++ | ++ |
| Example 2 | ++ | − |
| Comp. Ex. 2 | + | − |

(−): Less than 4 times the amount of antibody produced in Comparative Example 1, or the number of CTLs less than 30 cells/well
(+): 4 times or more and less than 8 times the amount of antibody produced in Comparative Example 1, or the number of CTLs 30 cells/well or more and less than 100 cells/well
(++): 8 times or more and less than 16 times the amount of antibody produced in Comparative Example 1, or the number of CTLs 100 cells/well or more and less than 300 cells/well
(+++): 16 times or more the amount of antibody produced in Comparative Example 1, or the number of CTLs 300 cells/well or more

[Synthesis of MOFs]

The MOFs shown in Tables 4 to 9 were prepared. Known substances among them were synthesized according to literature methods. The unreported substances were synthesized by hydrothermal treatment of the corresponding metal nitrate and the ligand in the presence of DMF.

[Evaluation of Adsorption Properties of MOFs]

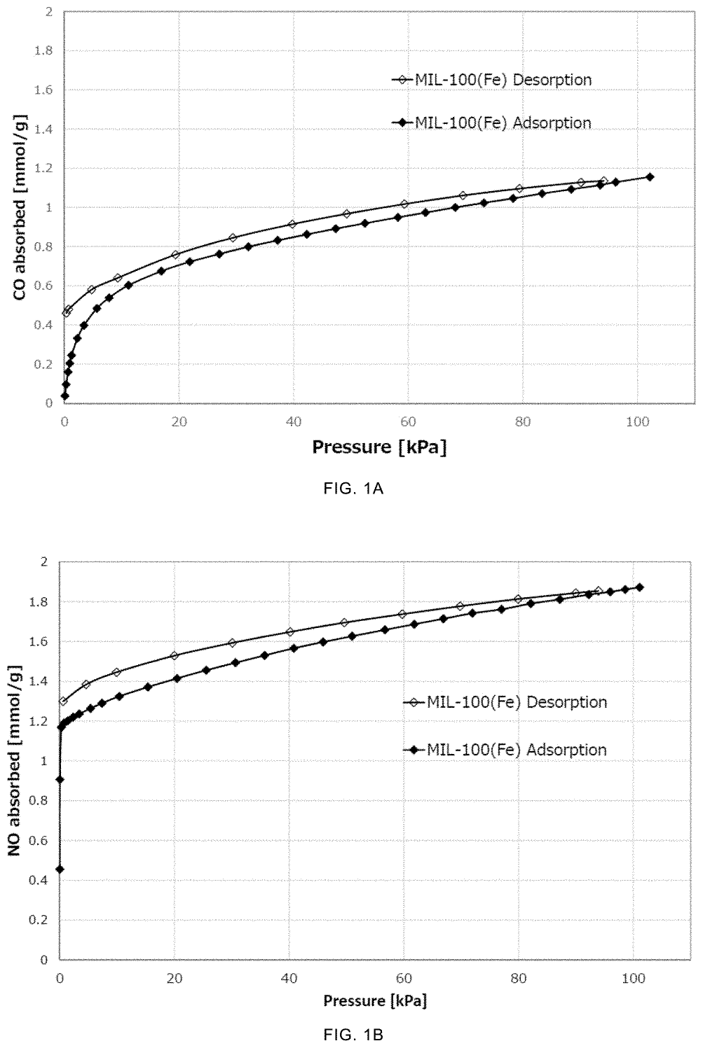

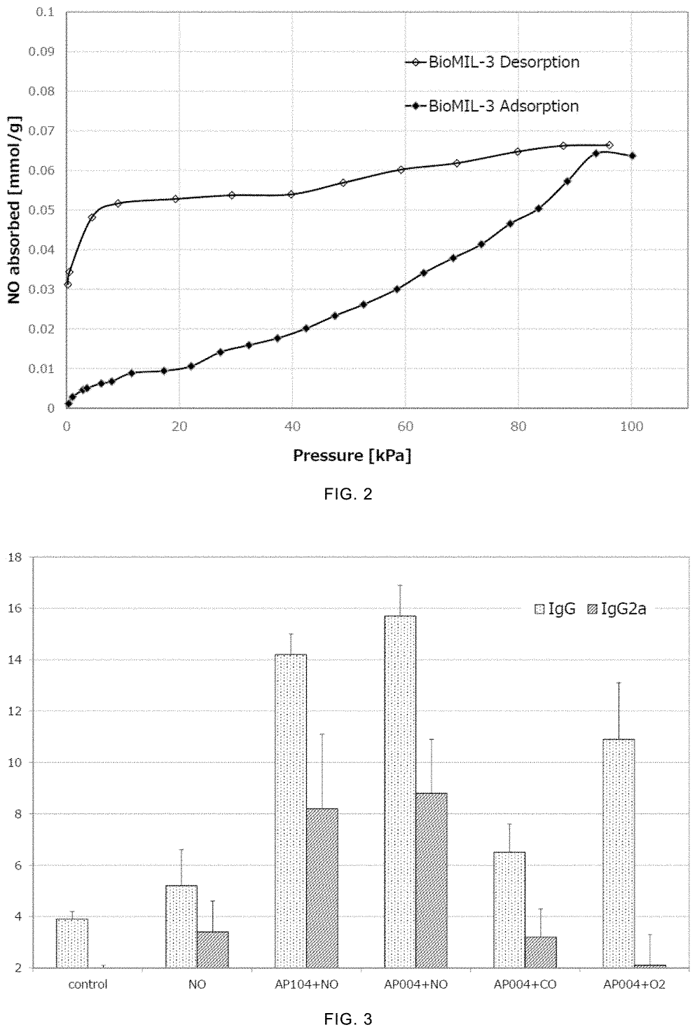

The amount of adsorption was measured by BELSORP-max12 (MicrotracBEL Co., Ltd.). The MOFs in powder form were used for the measurements. Some of the results are shown in FIG. 1A, FIG. 1B and FIG. 2 as representative examples. FIG. 1A is a CO adsorption profile of AP004 [MIL-100 (Fe)]. FIG. 1B is a NO adsorption profile of AP004 [MIL-100 (Fe)]. FIG. 2 is a NO adsorption profile of AP104 (BioMIL-3). In these examples, the adsorption/desorption profiles were irreversible. That is, when seen at the same pressure, the guest amount at the time of desorption was larger than the guest amount at the time of adsorption. Also, the residual amount of the guest in the MOFs were non-zero after performing the adsorption process from a vacuum state to a pressurized state and then performing the desorption process from the pressurized state to the vacuum state.

[Introduction of Immune Signal Transducers into MOFs]

In some of the examples below, the MOFs to which an immune signal transducer had been introduced were employed. Specifically, the degassing was performed by heating the MOF under a nitrogen flow. The sample was then returned to a room temperature and was exposed to an immune signal transducer. In particular, when the immune signal transducer was a gas, the sample returned to room temperature was exposed to a gas flow. A nitrogen flow was then performed at room temperature to discharge excess immune signal transducer. In this way, a MOF compound to which an immune signal transducer had been introduced was obtained.

The existence of the immune signal transducer in the MOF was checked by heating the sample under nitrogen flow and detecting the released immune signal transducer by a detector tube. It was thus confirmed that the immune signal transducer had effectively been introduced into the MOFs.

[Mouse Immunity Test]

An injection having the composition shown in Table 19 below was prepared. Specifically, the antigen and the MOF were weighed out in the amounts specified in Table 19, and glycerin was added thereto and mixed to obtain a vaccine composition. In the table, MOF stands for Metal Organic Framework and Gly for glycerin. In some examples, MOFs adsorbed with an immune signal transducer were used.

TABLE 19

| | | | | | | Antigen | | | | | | |
|---|---|---|---|---|---|---|---|---|---|---|---|---|
| Compounds | | | | | | (OVA) | | | | The num- | | |
| | | Immune | | Concen- | Concen- | Concen- | | | Subcutaneous | ber of | Evaluation | |
| | | Signal | Molecular | tration | tration | tration | Sol- | n- | injection | adminis- | Antibody | Cytokine |
| MOF | | Transducer | Weight | [μmol/mL] | [μg/mL] | [μg/mL] | vent | number | [μL/time] | trations | titer | (Spleen) |
| — | | | | — | — | 50 | Gly | 6 | 50 | 2 | IgG | IL-4 |
| AP104 | BioMIL-3 | — | 434 | 1 | 434 | 50 | | | | (2 wks) | IgG2a | IFN-γ |
| | | NO | | | 0 | 50 | | | | | | |
| AP004 | MIL-100(Fe) | — | 679 | 1 | 679 | 50 | | | | | | |
| | | NO | | | | 50 | | | | | | |
| | | CO | | | | 50 | | | | | | |
| | | O₂ | | | | 50 | | | | | | |

Using a liquid prepared as described above, 50 μL of an injection was administered subcutaneously to the back of a mouse (BALB/c mouse, female 7 weeks old). Two weeks after the administration, the same administration was again performed subcutaneously on the back of the mouse.

Two weeks after the second administration, mouse serum and spleen cells were collected, and serum OVA-specific IgG antibody and IgG2a antibody were measured by ELISA. In addition, the spleen cells were used to simultaneously evaluate the production amounts of OVA-specific IFN-γ and IL-4. The specific evaluation method is as follows.

[Measurement of Antigen-Specific Antibody Titer in Mouse Serum (ELISA method)]

As an antigen, an OVA-containing solution diluted with a carbonate buffer (100 μg/mL) was prepared. 100 μL of the solution was added to each well of a 96-well plate for ELISA and allowed to stand overnight.

The wells were washed 3 times with a washing solution (PBS containing Tween 20). 200 μL of a blocking solution obtained by diluting a blocking agent (Block Ace, Sumitomo Dainippon Pharma Co., Ltd.) with purified water to 4 g/100 mL was added and was left for 2 hours at room temperature. The wells were washed three times with the washing solution again.

Serum collected from a mouse in advance was centrifuged at 3000 g for 10 minutes at 4° C., and the obtained supernatant was collected. The above-mentioned supernatant was serially diluted to two times using a solution obtained by diluting the blocking agent with a phosphate buffer (Nacalai Tesque) to 0.4 g/100 mL. 50 μL of each of the obtained diluted solutions was added and left at room temperature for 2 hours.

The wells were washed three times with the washing solution once again. An HRP-labeled anti-mouse IgG antibody (Goat-anti mouse IgG Fc HRP, BETHYL) or an HRP-labeled anti-mouse IgG2a antibody (Goat-anti mouse IgG2a Fc HRP, BETHYL) was diluted to 10000 times with the solution obtained by diluting the blocking agent with a phosphate buffer (Nacalai Tesque) to 0.4 g/100 mL. 100 μL of each of the obtained diluted solutions was added and left at room temperature for 1 hour.

The wells were washed three times with the washing solution, and 100 μL of TMB solution (ELISA POD TMB kit, Nacalai Tesque) was added, and was left in the dark for 30 minutes.

Further, 100 μL of 1M sulfuric acid solution was added, and the absorbance at 450 nm was measured for each of the 96-well plates using a microplate reader. Based on the absorbance of the serially diluted samples, the IgG antibody titer or the IgG2a antibody titer in mouse serum was determined by Log 2 scale.

These results are summarized in FIG. 3. As shown in FIG. 3, it was revealed that the immune properties could be controlled by use of MOFs. Also, it was made clear that the immune characteristics could be further changed by a combination of MOF and immune signal transducer.

[Measurement of OVA-Specific Cytokine Production (ELISA method)]

100 μL each of 4×10⁵ cells/well of spleen cells collected in advance from a mouse was added to a 96-well plate for ELISA. 100 μL of an OVA-containing solution (100 μg/m) diluted in RPMI medium was thereto added and allowed to stand for 72 hours. The culture supernatant was collected, and the amount of each cytokine produced was quantified using a mouse IFN-γ ELISA kit and mouse IL-4 ELISA kit (R&D Systems).

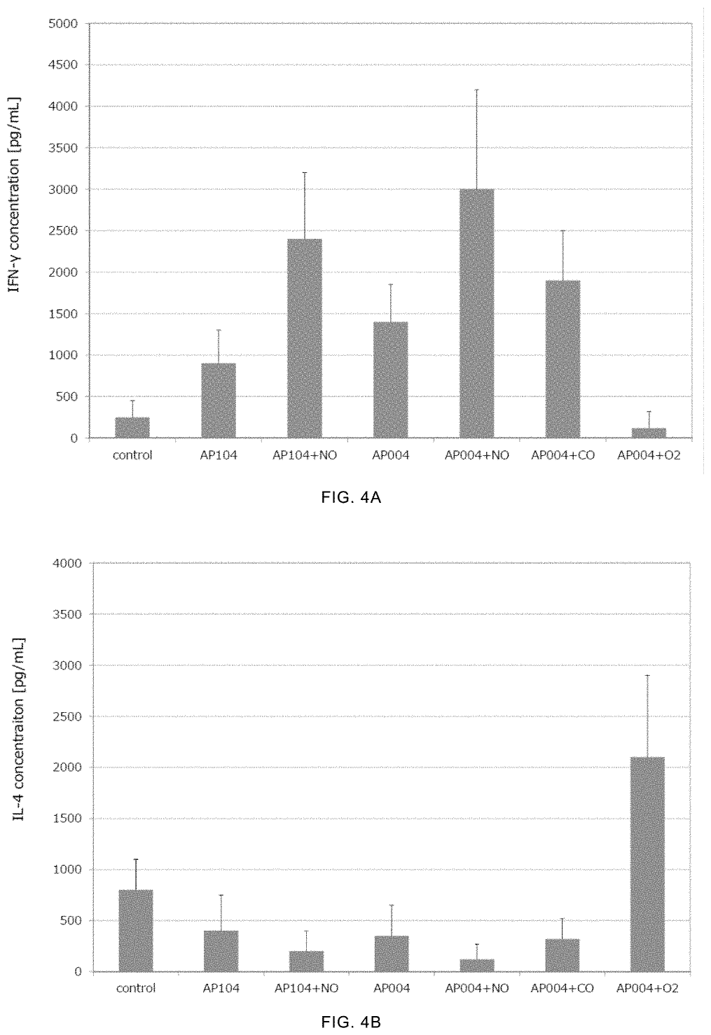

These results are summarized in FIGS. 4A and 4B. As shown in FIGS. 4A and 4B, it was revealed that the immune properties could be controlled by use of MOFs. Also, it was made clear that the immune characteristics could be further changed by a combination of MOF and immune signal transducer.

1. A vaccine composition comprising an antigen for inducing immunity and a Metal Organic Framework (MOF).

2. The vaccine composition according to claim 1, further comprising an immune signal transducer.

3. The vaccine composition according to claim 1, wherein at least a part of the immune signal transducer is contained in pores of the MOF.

4. The vaccine composition according to claim 3, wherein the MOF is configured to decompose in vivo to release at least a part of the immune signal transducer.

5. The vaccine composition according to claim 2, wherein the immune signal transducer is a small molecule having a molecular weight of 1000 or less.

6. The vaccine composition according to claim 5, wherein the immune signal transducer is a gas at 25° C. and 100 kPa.

7. The vaccine composition according to claim 2, wherein the immune signal transducer is a factor that is configured to act on keratinocytes, monocytes, lymphocytes, or granulocytes.

8. The vaccine composition according to claim 1, wherein the MOF comprises at least one metal element selected from the group consisting of calcium, magnesium, iron, zinc, aluminum, potassium, and sodium.

9. The vaccine composition according to claim 1, wherein the vaccine composition is configured to be administered on a skin and/or a mucous membrane.

10. The vaccine composition according to claim 1, wherein the vaccine composition is configured to be administered by an intradermal injection, a subcutaneous injection, or an intramuscular injection.

11. An adjuvant comprising a Metal Organic Framework (MOF).

12. The adjuvant according to claim 11, wherein the MOF contains an immune signal transducer in pores of the MOF.

13. The adjuvant according to claim 12, wherein the MOF is configured to decompose in vivo to release at least a part of the immune signal transducer.

14. The vaccine composition according to claim 3, wherein the immune signal transducer is a small molecule having a molecular weight of 1000 or less.

15. The vaccine composition according to claim 4, wherein the immune signal transducer is a small molecule having a molecular weight of 1000 or less.

16. The vaccine composition according to claim 14, wherein the immune signal transducer is a gas at 25° C. and 100 kPa.

17. The vaccine composition according to claim 15, wherein the immune signal transducer is a gas at 25° C. and 100 kPa.

18. The vaccine composition according to claim 2, wherein the immune signal transducer is a factor that is configured to act on keratinocytes, monocytes, lymphocytes, or granulocytes.

19. The vaccine composition according to claim 3, wherein the immune signal transducer is a factor that is configured to act on keratinocytes, monocytes, lymphocytes, or granulocytes.

20. The vaccine composition according to claim 4, wherein the immune signal transducer is a factor that is configured to act on keratinocytes, monocytes, lymphocytes, or granulocytes.
